(19) United States
(12) Patent Application Publication
(10) Pub. No.: US 2007/0027365 A1
(43) Pub. Date: Feb. 1, 2007

(54) A Method and System of After Hours Monitoring of a veterinary patient

(75) Inventor: Dale Herman Kosted, Enid, OK (US)

Correspondence Address: Dale Kosted, D.V.M., 3502 King St., Enid, OK, 73703, US

(73) Assignee: Dale Herman Kosted, Enid, OK (US)

(21) Appl. No.: 11/161,287

(22) Filed: Jul. 28, 2005

(51) Int. Cl. A61B 5/00

(52) U.S. Cl. 600/300

(57) ABSTRACT

After Hours Monitoring is a method of use system for monitoring a veterinary patient comprising: a monitor which is capable of receiving signals of low-power radio frequencies of physiological data from multiple wireless sensors; a remote surveillance system for visual observation of said patient.

### FIELD OF THE INVENTION

The present invention relates generally to uses of sensors/transmitters and a receiver device and an audio/video transmitter and receiver device, and more particularly to a method of use system for monitoring a veterinary patient after hours.

### BACKGROUND OF THE INVENTION

In the prior art many sensor/transmitters and receivers and many audio/video transmitters and receivers have been described. And, in the prior art systems for continually monitoring a patient have been disclosed and systems for surveillance have been disclosed. Many patient monitoring devices have been designed with various functions to aid in monitoring a patient, and they have been widely described in the Prior Art. See for example U.S. Pat. Nos. 6,733,447; 5,619,991; 5,664,270; 5,678,562; 5,704,366; 5,718,234; 5,735,285; 5,735,285; 5,754,111; 5,767,791; 5,827,180; 5,944,659; 5,961,448; 6,319,200. However, there are no systems described in the prior art which makes novel use of these systems separately or in combination to effectively and completely monitor a veterinary patient as does this present invention.

In the past, until this present invention, the problem has been one of economics. Providing twenty-four hour human monitoring of a veterinary patient is too expensive or cost prohibitive. As a result, many critical care veterinary patients are only monitored intermittently or not at all during the periods a facility is closed and not staffed. Therefore, an economical need exists for a method of use system to continually monitor visually and physiologically a veterinary patient.

### SUMMARY OF THE INVENTION

An object of the present invention is to provide a method of use system for After Hours Monitoring of a veterinary patient by allowing the user to observe audio/visual and physiological signs of a the patient. After Hours Monitoring is a method of use system for monitoring a veterinary patient comprising: 1.) a monitor, which interfaces with conventional PC platforms, capable of receiving low-power radio frequencies from multiple wireless sensor/transmitters of physiological data; software with file transfer protocol (FTP) allowing a user with an internet, intranet, LAN, connection from any location worldwide to access a secure, password protected website and monitor, in real-time, the veterinary patient, and allowing the user to set upper and lower limits for each sensor transmitter placed in or on a veterinary patient which will activate a notification method selected by the user if these limits are exceeded or subceeded; multiple, wireless sensors which transmit low-power radio frequencies of known physiological data; 2.) a surveillance system connected to the same PC via a USB video grabber capable of receiving wireless audio/video transmissions from wireless audio/video cameras/transmitters; wireless audio/video cameras/transmitters mounted in or within close proximity of the cage of a veterinary patient; an audio/video wireless receiver; specifically designed surveillance software with a USB video grabber capable of: broadcasting live video; allowing a user to simultaneously view, broadcast, and record multiple cameras via a PC internet, intranet, or LAN connection, from any location worldwide; recording live or when motion is detected; sends a notification if motion is detected; password protected to prevent unauthorized usage; scheduling for automatic viewing or recording; allowing a user to define independent motion detection zones for each wireless camera transmitter.

### BRIEF DESCRIPTION OF THE INVENTION

After Hours Monitoring is a method of use system for monitoring a veterinary patient comprising:

1.) a monitor: capable of receiving signals of low-power radio frequencies of physiological data from multiple wireless sensors; capable of interfacing with conventional PC platforms; specifically designed software with file transfer protocol (FTP) allowing a user with an internet, intranet, LAN, connection from any location worldwide to access a secure, password protected website and monitor, in real-time, the veterinary patient, and allowing the user to set upper and lower limits for each sensor transmitter placed in or on a veterinary patient which will activate a notification method selected by the user if these limits are exceeded or subceeded; multiple, wireless sensors which transmit low-power radio frequencies of known parameters of physiological data, not limited too: core body temperature (CBT); external body temperature; heart rate; breathing rate; pO2; CO2; 2.) a remote surveillance system comprising: wireless audio/video camera transmitters mounted in or within close proximity of the cage of a veterinary patient; an audio/video wireless receiver; specifically designed surveillance software with a USB video grabber capable of: broadcasting live video; allowing a user to simultaneously view, broadcast, and record multiple cameras via a PC internet, intranet, or LAN connection, from any location worldwide; recording live or when motion is detected; sends a notification if motion is detected; password protected to prevent unauthorized usage; scheduling for automatic viewing or recording; allows user to define independent motion detection zones for each wireless camera transmitter.

### DETAILED DESCRIPTION OF THE INVENTION

The method of the present invention is as follows:

1.) wireless sensor transmitters are initialized and activated then placed in or on a veterinary patient;

2.) wireless audio/video cameras/transmitters are activated.

The system comprises:

1.) a monitor: capable of receiving signals of low-power radio frequencies of physiological data from multiple wireless sensors; capable of interfacing with conventional PC platforms;
2.) specifically designed software with file transfer protocol (FTP) allowing a user with an internet, intranet, LAN, connection from any location worldwide to access a secure, password protected website and monitor, in real-time, the veterinary patient, and allowing the user to set upper and lower limits for each sensor transmitter placed in or on a veterinary patient which will activate a notification method selected by the user if these limits are exceeded or subceeded;
3.) multiple, wireless sensors which transmit low-power radio frequencies consisting of two types: a capsule shaped sensor which can be ingested or inserted rectally; dermal patch sensors which are placed on the skin.
4.) a remote surveillance system comprising: wireless audio/video camera transmitters mounted in or within close proximity of the cage of a veterinary patient; an audio/video wireless receiver; specifically designed surveillance software with a USB video grabber capable of: broadcasting live video; allowing a user to simultaneously view, broadcast, and record multiple cameras via a PC internet, intranet, or LAN connection, from any location worldwide; recording live or when motion is detected; sends a notification if motion is detected; password protected to prevent unauthorized usage; scheduling for automatic viewing or recording; allows user to define independent motion detection zones for each wireless camera transmitter

What is claimed is:

1. a method and system of After Hours Monitoring of a veterinary patient comprising:
a monitor: capable of receiving signals of low-power radio frequencies of physiological data from multiple wireless sensors; capable of interfacing with conventional PC platforms; with specifically designed software with file transfer protocol (FTP) allowing a user with an internet, intranet, or LAN connection from any location worldwide to access a secure, password protected intranet or internet website and monitor, in real-time, a veterinary patient, and allowing a user to set upper and lower limits for each sensor transmitter placed in or on a veterinary patient which will activate a notification method selected by said user if these limits are exceeded or subceeded;
a remote surveillance system comprising: wireless audio/video camera transmitters mounted in or within close proximity of the cage of a veterinary patient; an audio/video wireless receiver; specifically designed surveillance software with a USB video grabber capable of: broadcasting live video; allowing a user to simultaneously view, broadcast, and record multiple cameras via a PC internet, intranet, or LAN connection, from any location worldwide; recording live or when motion is detected; sends a notification if motion is detected; password protected to prevent unauthorized usage; scheduling for automatic viewing or recording; allows user to define independent motion detection zones for each wireless camera transmitter.

2. said monitor of claim 1 initializes and activates said multiple wireless sensors.

3. the multiple wireless sensors of claim 1 are of two types:
1.) a capsule shaped sensor;
2.) adhesive dermal patch sensors.

4. said capsule shaped sensor of claim 3 can be ingested or inserted rectally.

5. said adhesive dermal patch sensors of claim 3 are placed on the skin of a veterinary patient.

6. said notification methods of claim 1 can include, but not limited to: an alarm bell; an alarm buzzer; an alarm ringer; an alarm horn; a telephone call; an e-mail; a pager message; a text message; a fax; any future developed method of notification.

7. said monitor software of claim 1 is capable of analyzing said physiological
data transmissions received from the multiple wireless sensors.
